(19) United States
(12) Patent Application Publication
(10) Pub. No.: US 2017/0274405 A1
(43) Pub. Date: Sep. 28, 2017

(54) Portable Diffuser

(71) Applicants: David Kalim Lucas, Singapore (SG); Paymon Rasekhy, Singapore (SG); Andrew Riaz Lucas, Singapore (SG); Virasawmy Davissen, Singapore (SG)

(72) Inventors: David Kalim Lucas, Singapore (SG); Paymon Rasekhy, Singapore (SG); Andrew Riaz Lucas, Singapore (SG); Virasawmy Davissen, Singapore (SG)

(73) Assignee: Lynxemi Pte. Ltd., Singapore (SG)

(21) Appl. No.: 15/417,200

(22) Filed: Jan. 26, 2017

Related U.S. Application Data
(60) Provisional application No. 62/291,501, filed on Feb. 4, 2016.

(51) Int. Cl. B05B 17/00; A61L 9/14; B05B 17/06; H01M 10/0525; H01M 10/0565

(52) U.S. Cl. CPC B05B 17/0646; H01M 10/0525; H01M 10/0565; B05B 17/0661; B05B 17/0684; A61L 9/14; H01M 2300/0082; H01M 2220/30; A61L 2209/11; A61L 2209/132

(57) ABSTRACT

The complete system for a portable diffusing device, including diffusing engine, housing, delivery of active materials, active material reservoir container, power source, user interface with the device, as well as a case enclosing the device, is described in this invention. The device incorporates a piezoelectric dispersion mechanism with a staged active material delivery mechanism, allowing for ease of removal and/or replacement of the active material reservoir container via a simple means of attachment or detachment. The invention can comprise either one complete system housed in one device, or a multiplicity of systems housed in one device. The device of the invention also incorporates an on-board power source allowing for portability of the device, as well as a direct interface mechanism and/or a wireless mechanism for user control of the device. The invention also includes a case for housing, charging, and communicating with the device.

### CROSS REFERENCE TO RELATED APPLICATION

This application claims priority under 35 U.S.C. §119 to U.S. Provisional Application No. 62/291,501, filed 4 Feb. 2016, the contents of which are incorporated herein by reference.

### TECHNICAL FIELD

The present disclosure relates to a portable diffusing technology for use with liquid active materials, with particular regards to essential oils, fragrance oils, water-based fragrances, and the like. The technology can also be used for applications involving portable aerosolization e.g., as a portable humidifier, for medicine delivery, as an insect repellant, and so on.

### BACKGROUND

The dispersing of active materials (with particular regards to fluid-based active materials) as aerosols is important for a number of applications, including, but not limited to, air fresheners, medical drug delivery systems, fuel systems, analytical instrumentation, insect repellants, hygiene maintenance systems, and so forth. There are a multitude of active material diffusion devices available in the market, all of which use different means for dispersion. For example, some diffusion devices include a heating element for heating an active material in order to evaporate the material. Other diffusion devices utilize an air stream via a fan or an air pump to evaporate and/or stream evaporated active material from the diffusion device into the environment. Other active material diffusion devices dispense active material utilising an ultrasonic disperser. Many of these devices are passive, in that only ambient air flow is required to disperse the active material therein (either liquid, wax-based, microcapsules, or otherwise). Other devices require an active dispersing engine, and are either battery-powered or receive external power e.g., via a cord extending from the device plugged into an external socket.

An example of a common type of active material used in dispersion devices is essential oil. Essential oils are used for a range of purposes including air freshening and medicinal applications. Often these oils are usually mixed with water and diffused, or are heated with a candle and evaporated. Many diffusing devices for essential oils are known and are commercially available, including, but not limited to, reed diffusers, water-based aroma humidifiers, wax-based diffusers, and air-pump devices. Many of these types of devices are used for aromatherapy and air freshening. An example of a common type of aromatherapy or air freshening diffuser uses heat to diffuse the active material. Such heat-based diffusers usually require a heat source such as a candle flame or an electric element to evaporate the essential oil/fragrance, but this degrades the quality of the oil and may also pose both smoke inhalation and fire hazards. Aspirator-based diffusers (e.g., that utilize an air pump) also require external source of power due to the large energy consumed by the pump and/or vibrating motor. Examples of patents for essential oil diffusers U.S. Pat. Nos. 8,066,420 and 8,147,116, where essential oils are either directly heated or added to water and then dispersed.

Piezoelectrically-driven liquid atomization apparatus are described in various examples of prior art, including but not limited to, U.S. Pat. Nos. 6,293,474, 6,341,732, 6,382,522, 6,450,419, 68,434,130, 7,469,844, 20070012718, 20140191063, 20140263722, and 20150117056. These patents each describe piezoelectrically-driven fluid atomization apparatus, usually comprising a piezoelectric driving engine comprised of ceramic piezoelectric material joined or connected to an atomization mechanism (e.g., plate, mesh, perforated metal film, and so forth). Driving the piezoelectric engine via an alternating current electrical voltage causes the atomization mechanism to atomize and disperse the fluid in a controlled and well-defined manner. In terms of fluid delivery to the piezoelectric engine, the fluid can be delivered using a variety of mechanisms, including but not limited to wicking, pumping, air pumping, capillary tubing, mesh capillary wicking, and so forth. Although it is not necessary to include complex electronic circuits apart from those needed to drive the piezoelectric engine, one is often attached with these inventions to provide both greater user control of these devices as well as the requesite electrical current to the various elements of the respective devices.

A number of air freshening diffusers are capable of diffusing multiple scents, for example, by using a cartridge containing a variety of encapsulated scent elements (comprised of encapsulated or wax-based essential oils, or derivatives thereof). In one example of this type of air freshener, an airflow generator (e.g., fan, pump, air jet, and so forth) is used to generate airflow across the receiving cartridge, and then flow out of an opening in the device housing. This type of device can diffuse one scent, and then, depending on the cartridge design, dispense a second scent when the cartridge position is changed. However, this type of device is usually limited to active materials that are solid, such as fragranced waxes. In terms of liquid active materials, there are other examples of diffusing devices that are capable of dispensing multiple liquid active materials from the same device, usually from different dispersing ports. In one example of this type of device, there are three active material liquid reservoirs, and the active material liquid in each reservoir is transmitted to a piezoelectric engine via a wick and dispersed into the environment by ultrasonic vibration. However, in this example, replacement of the active material reservoir is not straightforward, and it is preferred that the active material is fully consumed before the reservoir is replaced. As such, a need remains for device that allows users to disperse multiple active materials as required, while allowing for greater flexibility and greater choice regarding what material to disperse and how to replace each material without necessarily consuming all the active material in the device's reservoirs prior to replacement.

Importantly, many of the types of diffuser devices such as the examples given above require a significant amount of energy to operate, and thus need an external power source to function; consequently, apart from a few exceptions (e.g., U.S. Pat. No. 6,802,460), these diffusers do not usually have an on-board power source such as a replaceable or rechargeable battery. Additionally, while some of the aforementioned diffusers can disperse multiple scents (e.g., U.S. Pat. No. 7,469,844), replacement or replenishment of the active material reservoir or reservoirs in many commercially available devices is usually difficult to undertake if it is possible at all, and usually requires disassembly and cleaning of the requisite components. While some commercially available essential oil dispersers allow the user to change the dispersing fluid, these devices often require that all of the fluid previously loaded into the device be consumed prior to refilling the reservoir, which reduces the flexibility of the device for the device operator. Furthermore, a number of commercially available devices are designed so that the user cannot easily disassemble the device and access or replenish the fluid reservoir, thus rendering the device disposable in its entirety and adding to cost and waste.

Note that the content of documents referred to in this application are incorporated by reference to this application, for the purpose of providing examples for comparison only.

### SUMMARY OF THE INVENTION

According to one aspect of the present invention, an apparatus for dispersing active materials comprises a housing that includes the dispersing engine, active material transfer mechanism, replaceable active material reservoir container(s) and associated transfer mechanisms, power source, and programmable driving circuitry. The circuitry includes a programmable microprocessor that can be interfaced with either via a direct mechanism (e.g., button interface, touch screen, movement sensor, USB cable attached to a computer or smartphone, and so forth) or via a wireless mechanism (e.g., from an external controller such as a computer or smartphone connected to the device via Bluetooth, IEEE 802.11 standards, and so forth). The circuitry and microprocessor provide a mode of operation that can be user-controlled, in that the device user can determine how the active material is dispensed via the direct interface and/or the wireless interface. The apparatus can be externally powered or powered via an on-board power source, which can either be replaced (if the power source is non-rechargeable) or recharged (if the power source is rechargeable) via a direct mechanism e.g., USB cable, power cable, or via an indirect mechanism e.g., wireless charging.

According to another aspect of the present invention, the active material can be connected to or disconnected from the dispersing apparatus via a simple step of adding or removing the active material reservoir container. The active material reservoir container is designed so that the active material can be readily transported to the piezoelectric dispersing engine via a multi-staged transfer mechanism. In a preferred embodiment, the active material is transferred via a two-stage mechanism. The mode of attachment of the reservoir to the dispersing apparatus can be via using magnets, clips, adhesives, screws, bands, and so forth. In a preferred embodiment, the mode of attachment is via magnets.

In another aspect of the present invention, the number of diffusing systems, comprising piezoelectric diffusing apparatus, transfer mechanisms, and reservoirs, can be greater than one. In one embodiment, the diffusing apparatus and reservoirs are housed as discrete modules in one housing, thus allowing for more than one dispersing system to be contained in one device. In a further embodiment, each dispersing system can be controlled, either as a single and separate dispersion system or together as multiple dispersion systems operating simultaneously, via a single interface that is either a direct mechanism (e.g., button interface, touch screen, movement sensor, USB cable attached to a computer or smartphone, and so forth) or a wireless mechanism (e.g., from an external controller such as a computer or smartphone connected to the device via Bluetooth, IEEE 802.11, and so forth). In a preferred embodiment, there are three identical dispersing systems, including three diffusing apparatus, three transfer mechanisms, and three reservoirs, in one device, with one power source and driven by programmable driving circuitry, all controllable via a direct mechanism and/or wireless interface. The device can be externally powered or powered via an on-board power source, which can either be replaced (if the power source is non-rechargeable) or recharged (if the power source is rechargeable) via a direct mechanism e.g., USB cable, power cable, or via an indirect mechanism e.g., wireless charging.

In a further aspect of the invention, the device of the present invention can be housed in a case. In a preferred embodiment, the case comprises a bottom cover and a top cover that can be joined together to securely house the single system embodiment, with a connection cable provided with the case for ease of user access in order to charge and communicate with the device as needed. The connection cable can feature connecting ports that are compatible with the device housed within the case and with the external charging and communication station.

### BRIEF DESCRIPTION OF THE DRAWINGS

FIG. 1 is an oblique elevation view of the single system embodiment of the present invention, comprising one diffusing system;

FIG. 2 is a cutaway cross-section view of the embodiment shown in FIG. 1, with attached active material reservoir container within;

FIG. 3 is an upper-elevation exploded view of the top section of the embodiment shown in FIG. 1, showing important features of this section;

FIG. 4 is a lower-elevation exploded view of the top section of the embodiment shown in FIG. 1, showing important features of this section;

FIG. 5 is an exploded view of the bottom section of the embodiment shown in FIG. 1, with active material reservoir container not shown;

FIG. 6 is an oblique elevation view of the active material reservoir container;

FIG. 7 is an exploded view of the active material reservoir container shown in FIG. 6;

FIG. 8 is an oblique elevation view of a multi-system embodiment of the present invention, comprising three diffusing systems, as assembled in one housing;

FIG. 9 is an upper-elevation exploded view of the top section of the embodiment shown in FIG. 8, showing important features of this section;

FIG. 10 is a lower-elevation exploded view of the top section of the embodiment shown in FIG. 8, showing important features of this section;

FIG. 11 is an exploded view of the bottom section of the embodiment shown in FIG. 8, with active material reservoir containers not shown;

FIG. 12 is an oblique elevation view of a case embodiment that can enclose the single system embodiment of the present invention shown in FIG. 1;

FIG. 13 is an exploded view of the case embodiment of FIG. 12, showing the single system embodiment of FIG. 1 housed within and the USB charging and communication cable opened;

FIG. 14 is a lower-elevation exploded view of the case shown in FIG. 12, with USB charging and communication cable closed;

FIG. 15 is an upper elevation exploded view of the case shown in FIG. 12, with USB charging and communication cable opened.

### DETAILED DESCRIPTION

With reference to the following detailed description, the invention can be better understood when it is considered in conjunction with the drawings accompanying this application. Furthermore, other aspects and advantages of the device of the present invention are given in the following detailed description.

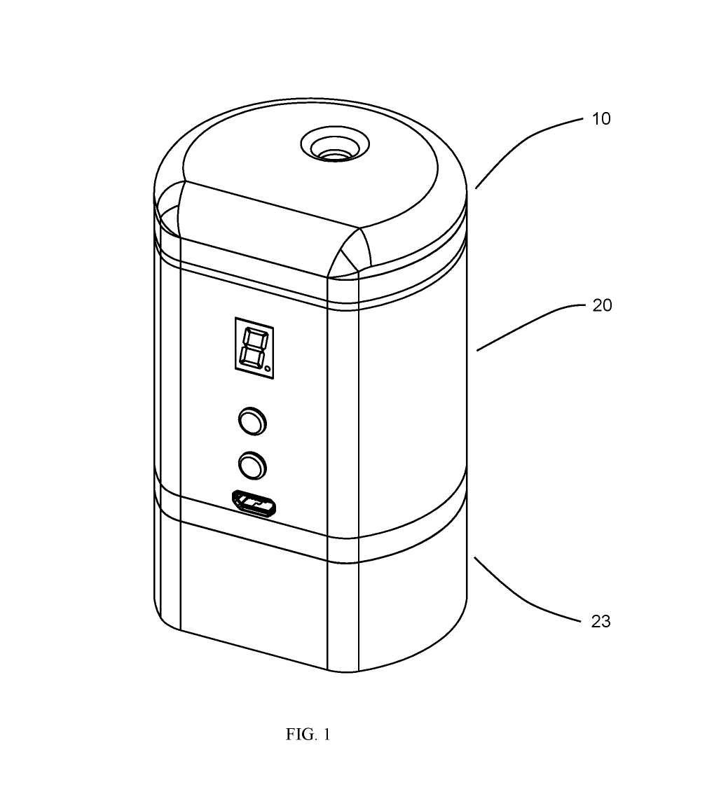

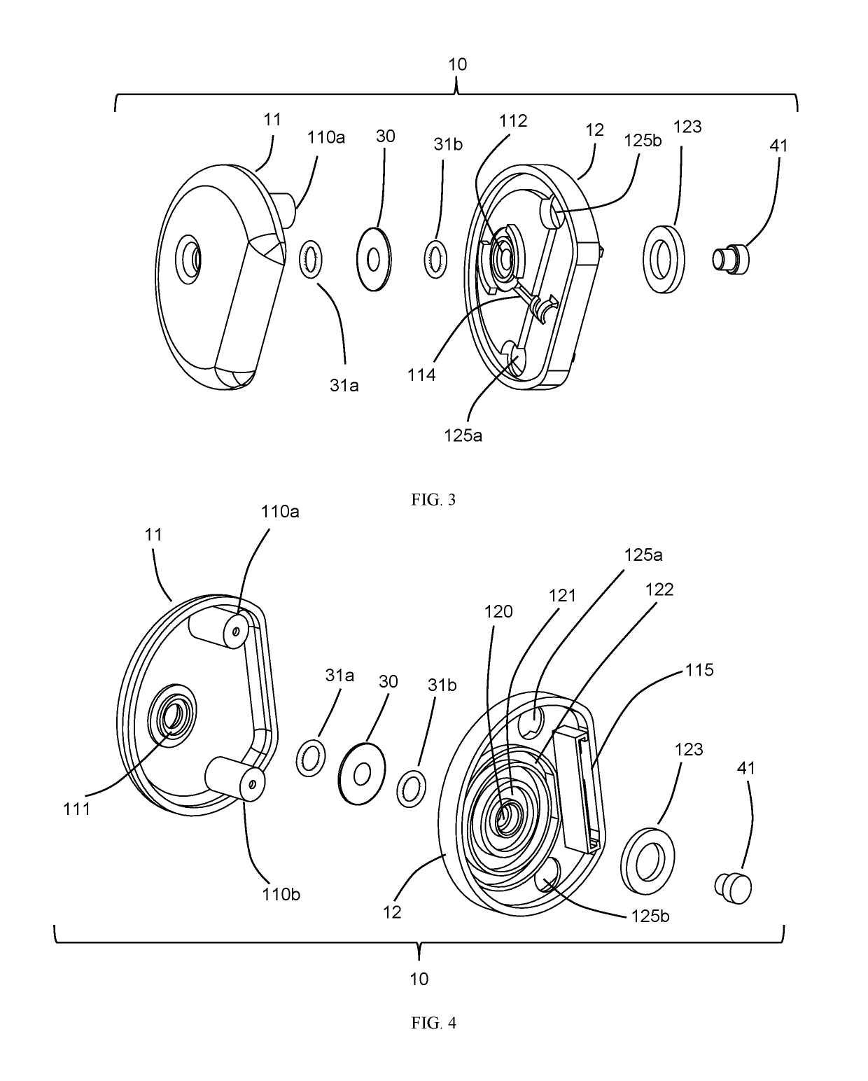

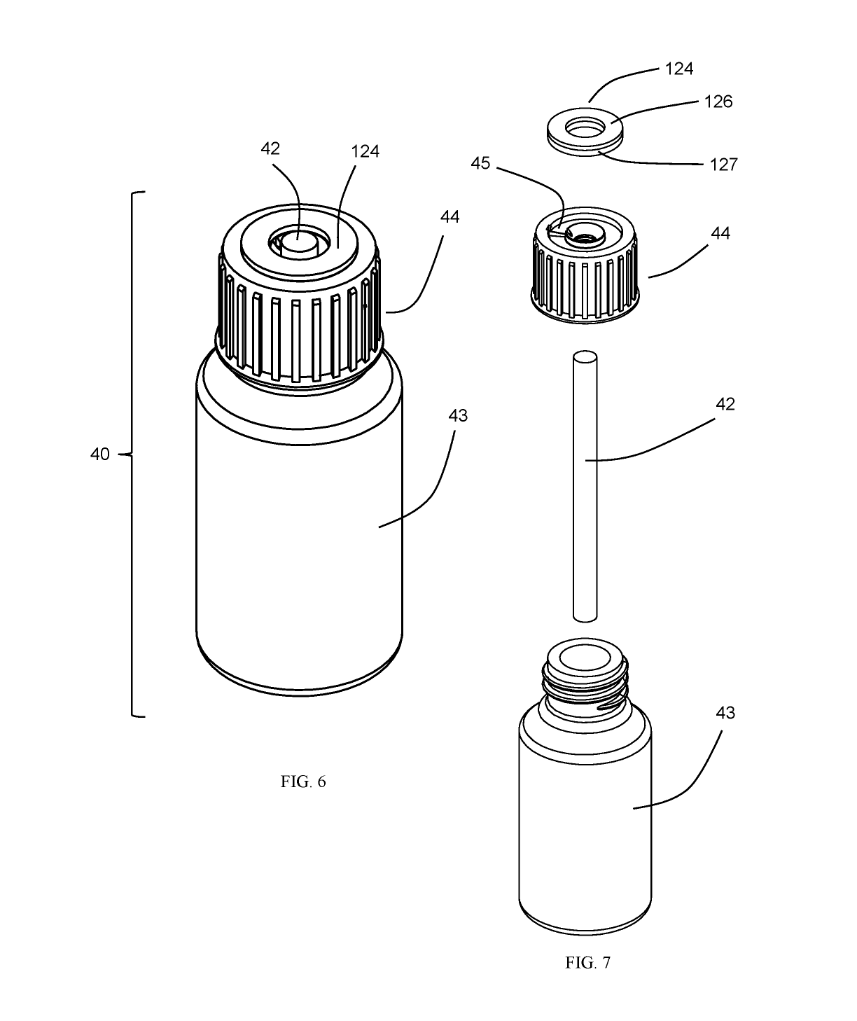

With reference to FIGS. 1 to 5, the present invention relates to using an ultrasonic piezoelectric diffusing engine (30) to finely disperse fluid delivered via a multi-staged delivery mechanism (41 and 42), along with the controlling electronics (220), power source (223), and design features of the device housing and integrated components (FIGS. 3 to 5). The device of the present invention can be used to dispense an active material over a period of time determined by the user, for which the user can adjust the frequency of dispersion and mode of operation. Furthermore, any active material reservoir containers (40, and FIGS. 6 and 7) placed into the device can be easily removed and replaced by the user.

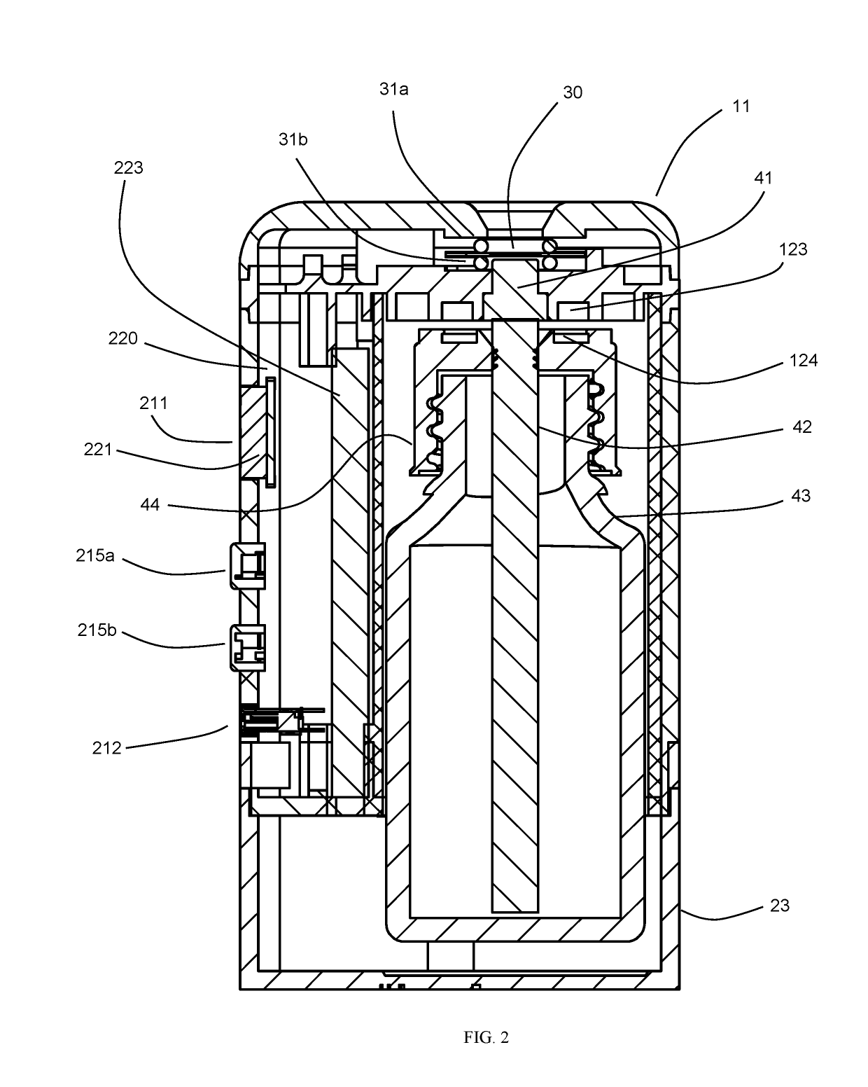

With reference to FIGS. 2, 3 and 4, the invention refers to the assembly of a piezoelectric diffusing engine (30) in contact with or very near to an active material fluid delivery component (41 e.g., wick, capillary, flow tube, dish, and the like). In a preferred embodiment, the piezoelectric diffusing engine (30) comprises a ceramic ring enclosing a perforated metal plate, film, or mesh, so that active material fluid can be captured by the metal plate, film, or mesh, and dispersed in aerosolized form into the environment from one or both sides of the metal plate, film, or mesh.

The piezoelectric diffusing engine (30) is mounted in contact with or near to one component of the active material fluid delivery system (41 and 42) in the invention housing (10, 20, and 23). The piezoelectric diffusing engine can be mounted using fixed brackets, clips, gaskets, toric rings (O-rings), glue, pivots, and so forth. In one embodiment, the mountings of the aforementioned types can have an ASTM D2240 type A hardness, also known as Shore A hardness, of between 10-90, preferably between 30-70, more preferably between 45-55.

In a preferred embodiment shown in FIGS. 2, 3, 4, and 5, the piezoelectric diffusing engine is mounted using top and bottom toric rings, also known as O-rings (31a and 31b). In this embodiment, the O-rings and piezoelectric engine are mounted between a top plate (11) of the top section (10) of the housing, which is also the top-most part of the device, and the bottom plate (12) of the top section (10) of the housing. The O-rings and piezoelectric engine fit into a top cavity (111) and a bottom cavity (112) in each of the plates. In a preferred embodiment, the plates are joined together with the middle housing section (20) by posts (110a and 110b) that slot into ports (125a and 125b) in the bottom plate (12), and are attached to the middle housing section by screws (113a and 113b) that are fitted through posts (213a and 213a) in the middle housing section (20). With regards to the O-rings of this embodiment (31a and 31b), the O-rings are comprised of a synthetic rubber material, examples of which include, but are not limited to, silicone, nitrile butadiene rubber (Nitrile), ethylene vinyl acetate copolymers (EVM), a fluoroelastomer comprising copolymers of hexafluoropropylene (HFP), vinylidene fluoride (VF2), polyvinylidene fluoride, terpolymers of tetrafluoroethylene (TFE), hexafluoropropylene (HFP) and VF2, perfluoromethylvinylether (PMVE), pentapolymers of TFE, HFP, ethylene, PMVE, and VF2, some combinations of which are commercially available as Viton A, Viton B, Viton F, and generally known as FKM, or, where the perfluoroelastomeric compounds contain an even higher amount of fluoride than the aforementioned combinations, FFKM. In a preferred embodiment, the O-rings are comprised of combinations of aforementioned compounds along with perfluoro(alkylvinyl) ether (PAVE) and either without additions of base-sensitive copolymers HFP and/or VF2 or with lower added concentrations of HFP and/or VF2, combinations of which are commercially available as Viton Extreme TBR-S, Viton Extreme ETP-600S, and Viton Extreme ETP-900.

The active material fluid delivery component (41) should be in contact with or very near to the piezoelectric diffusing engine, and comprises the last stage of the fluid delivery system. The delivery of fluid to the piezoelectric engine can be via a multiplicity of mechanisms, including, but not limited to, wicking, pumping, capillary action, air pumping, hydraulic action, and so forth. In a preferred embodiment, this last stage material comprises a hydrophilic, oleophilic, or both hydro- and oleophilic porous wick made of either natural fibers (e.g., cotton, silk, wood pulp, and the like) or synthetic fibers (e.g., polyethylene, polypropylene, polycarbonate, and the like). In a preferred embodiment, this stage of the fluid delivery material is a rigid, porous wick made of polyethylene. In a more preferred embodiment, this stage of the fluid delivery material is both hydrophilic and oleophilic, in that both water-based active materials and oil-based active materials can be transmitted through the material. In this more preferred embodiment, the rigid, porous, hydrophilic, and oleophilic wick (41) is fitted into a cavity (120) of the bottom plate (12) of the top section (10) of the device.

Regarding the staged delivery mechanism for the active material, specifically an active material fluid, there are various methods for inserting or removing active material reservoir containers into or out of the invention, and such containers may be easily removed and replaced by the user. With reference to FIGS. 3, 4, 5, 6, and 7, in one embodiment, the active material reservoir container (40, and FIGS. 6 and 7) is a container of specific volume from 1 mL to 10 L, preferably 1 mL to 1 L, more preferably 1 mL to 100 mL, and even more preferably 1 mL to 50 mL. In a preferred embodiment, the active material reservoir container is 30 mL. The base (43) of the active material reservoir container (40) can be made of any compatible storage material such as plastic, e.g., polypropylene (PP), polyethylene terephthalate (PET), polybutylene terephthalate (PBT), polycarbonate (PC), and so forth; or the storage material can be made of glass, e.g., silica, borosilicate, Pyrex, aluminosilicate, and so forth; or the storage material can be made of metal, e.g., stainless steel, aluminum, and so forth; or the storage material can be made of a combination of the aforementioned materials. The connection of the active material reservoir container base (43) to the device housing can be via screws, clips, magnets, glue, necked seal, and so forth. In a preferred embodiment, the active material reservoir container base (43) has a cap (44) that is screwed onto the reservoir container, which can be magnetically docked to the last stage of the fluid delivery system. In this preferred embodiment, a washer (124) is encased onto the active material reservoir container cap (44), which then docks with a ring-shaped magnet (123) that is housed in a cavity (121) situated in the bottom plate (12) of the top section (10) of the device, which also houses the last stage of the fluid delivery system, specifically the rigid, porous, hydrophilic, and oleophilic wick (41). Regarding the reservoir container cap (44), an airway is provided (45) in order to ensure uptake of active material by the device is not hindered by pressure differences between the active material storage container (40) and the outside environment, resulting from circumstances such as changes in the volume of active material in the container or changes in altitude. Regarding the washer embedded in the cap (124), in a preferred embodiment, this washer (124) is comprised of two parts, that is a metal washer (127) coated with a substance (126) in order to reduce leakage from the active material delivery interface, better seal the active material reservoir container (40) to the last stage of the fluid delivery mechanism (41 and 123), and prolong the lifetime of interface components. In a more preferred embodiment, the metal washer coating substance (126) is a synthetic rubber compound, and can be comprised of material similar to the materials used in the aforementioned O-rings, such as, but not limited to, silicone, nitrile butadiene rubber (Nitrile), ethylene vinyl acetate copolymers (EVM), or a fluoroelastomer product such as those commercially known as Viton A, Viton B, Viton F, and generally known as FKM, or, where the perfluoroelastomeric compounds contain an even higher amount of fluoride than the aforementioned combinations, FFKM, as well as commercially available perfluoroelastomers such as Viton Extreme TBR-S, Viton Extreme ETP-600S, and Viton Extreme ETP-900.

The delivery of the active material to the last stage of the fluid delivery mechanism can be a single stage or can involve multiple stages. With reference to FIGS. 2, 3, 4, 6, and 7, the delivery of fluid to the last stage (41) can be via a multiplicity of mechanisms, including, but not limited to, wicking, pumping, capillary action, air pumping, hydraulic action, and so forth. In one embodiment, this delivery mechanism (42) utilizes a single hydrophilic, oleophilic, or both hydro- and oleophilic porous wick made of either natural fibers (e.g., cotton, silk, wood pulp, and the like) or synthetic fibers (e.g., polyethylene, polypropylene, polycarbonate, borosilicate composite, and the like), to deliver fluid to the last stage. In a preferred embodiment, the wick (42) used for active material delivery is a soft, porous wick made of polyethylene that is both hydrophilic and oleophilic, in that both water-based active materials and oil-based active materials can be transmitted through the material.

The active material reservoir container (FIGS. 6 and 7) may contain one of or a combination of a range of active materials, including, but not limited to, a water-based fragrance, an oil-based fragrance, an essential oil, a mixture of essential oils, an insecticide, an insect repellant/attractant, a disinfectant, an air purification agent, an aromatherapy scent comprising oil-based and/or water-based scents, an odor eliminator, an air freshener, and so forth, or combinations thereof. Any combination of types of liquid active materials can be utilized in the active material reservoir container, and can be used with any of the embodiments disclosed in this application. The active material reservoir container can be replaced when empty or whenever the user requires a new active material.

With reference to FIGS. 3, 4, and 5, the operation of the piezoelectric diffusing engine can be undertaken via a connection to a printed circuit board (PCB, 220) and associated electronics, including microprocessor. The PCB can be situated in various places, including adjacent to the piezoelectric diffusing engine, or in a different part of the device housing. In a preferred embodiment, the PCB is mounted vertically along the back face of the middle section (20) of the device, and is supported by a placeholder (115) in the bottom plate (12) of the top section (10) of the device. The PCB can be connected to the piezoelectric engine (30) via wires that run along a channel (114) in the bottom plate (12) of the top section of the device (10). The piezoelectric engine is controlled by the microprocessor, which is in turn controlled by the user via a direct mechanism (e.g., button interface, touch screen, movement sensor, USB cable attached to a computer or smartphone, and so forth) or via a wireless mechanism (e.g., from an external controller such as a computer or smartphone connected to the device via Bluetooth, IEEE 802.11, and so forth). Information regarding the status of the device can be relayed to the user via a multiplicity of mechanisms, including, but not limited to, a segmented light-emitting diode (LED) display, a segmented liquid crystal display (LCD), an LCD, a touchscreen LCD, and/or via wired or wireless communication to an external device such as computer or smartphone. Regarding the wireless mechanism, the electronics of the device will include a transmitter to enable it to send and receive wireless signals. One preferred embodiment of the invention is able to send and receive signals using both Bluetooth and IEEE 802.11 standards in the 2.4, 3.6, 5 and 60 GHz frequency bands (Wi-Fi). This can allow the device to be controlled wirelessly, remotely, and securely e.g., via the Internet through a mobile smartphone communications network. In a preferred embodiment, the device may be controlled from a mobile smartphone via Bluetooth or Wi-Fi using a software application downloaded or purchased from an online application store. In another preferred embodiment, the device can also be turned off remotely using a wireless mechanism when operation of the device is not needed, e.g., if the device is located in a hotel room when the room is unoccupied.

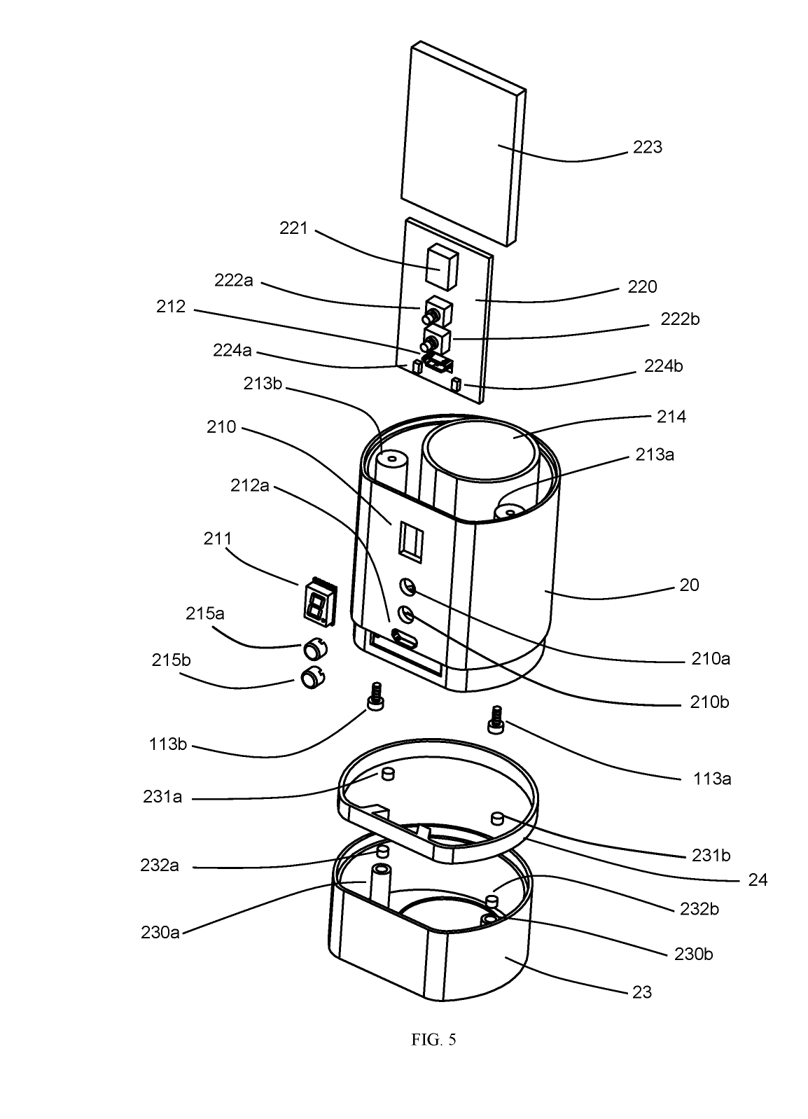

With reference to FIG. 5, the microprocessor is controlled via a direct mechanism that uses buttons (215a and 215b) fitted into ports (210a and 210b) in the middle section (20) to interface with switches (222a and 222b) on the PCB (220), along with a display interface (211 and 221). The device also features a receptacle (212) accessible via a port (212a) for the purpose of charging and/or communicating with the device, which can be of any appropriate charging and/or communication technology e.g., a USB standard (Type-A, Type-B, Mini-A, Mini-B, Micro-A, Micro-B, Type-C, and so forth), a DC connector (coaxial power connector, JSPB connector, mini-DIN connector, and so forth), an Ethernet port (Cat 5, Cat 5se, Cat 6, and so forth), a serial interface (RS232, IEEE 1394, and so forth), a Lightning connector, and any other means of charging and/or communication. In a preferred embodiment, the receptacle is a micro-USB (Micro-B) receptacle (212) accessible via the port (212a) in the middle section (20) of the device. Regarding the direct interface, in a preferred embodiment, two buttons (215a and 215b) are used in conjunction with a 7-segment light-emitting diode (LED) display (221) and a light pipe (211) fitted into a slot (210) in the middle section (20) to instruct the microprocessor to drive the invention according to the user's preference. When the device is in operation, two LEDs (224a and 224b) will activate. A ring-shaped light diffuser (24) transmits the light emitted by these LEDs (224a and 224b) as a glowing band around the perimeter of the device. The ring-shaped light diffuser (24) can be any color and can be clear, partially opaque, frosted, or opaque. In a preferred embodiment, the ring-shaped light diffuser (24) is made of frosted glass or frosted clear plastic such as frosted polyethylene, frosted polycarbonate, and the like.

An example of the operation of a preferred embodiment is given here; note that this example is for illustrative purposes only, and by no means limits the functionality of device in any way whatsoever. Importantly, the use of the device and the interface parameters can be customized, altered, changed, updated, and modified at any time by changing or updating the microprocessor program. In this illustrative example of a preferred embodiment, the user is able to turn the device on by holding the first button (215a, which activates switch 222a) firmly for 3 seconds. The device is switched on when the 7-segment display (221, transmitted to the outside of the device via a light pipe, 211, for the user to see) shows the characters ‘o’ and ‘n’ successively. When the device is on, the device can be turned off also by holding the first button (215a) firmly for 3 seconds, and the device is switched off after the 7-segment display (221) shows ‘o’, ‘F’, and ‘F’ successively. Once the device has been turned on, the user can set the run-time duration of the piezoelectric engine and the frequency of repeat run-time operations over a preconfigured time period, as set by the microprocessor program. The user enters the device configuration mode by holding the second button (215b, which activates switch 222b) for 2 seconds, after which the 7-segment display (221) shows ‘C’. The user is then able to set the operation of the piezoelectric engine by pressing the first button (215a), which cycles through three preset parameters, as shown on the 7-segment display: ‘t’ for oil type, ‘L’ for strength level, and ‘d’ for duration, all of which are settings that modify the run-time duration of the piezoelectric engine and time intervals between piezoelectric engine operations. When the 7-segment display (221) shows ‘t’ for oil type, the user can press the second button (215b) to select a preset active material liquid type on the basis of viscosity, numbered ‘1’ to ‘4’ as displayed on the 7-segment display (221). This setting configures the run-time duration of the piezoelectric engine, e.g. with a setting of ‘1’, the piezoelectric engine operates for 2 seconds, which is enough time to aspirate a lower-viscosity (e.g., viscosity between 0.5 to 1 centipoise) active material fluid, whereas a setting of ‘4’ operates the piezoelectric engine for 5 seconds, which is enough time to aspirate a higher-viscosity (e.g., viscosity between 3 to 4 centipoise) active material fluid. The user can move to the next setting, ‘L’ for strength, by pressing the first button (215a). When the 7-segment display (221) shows ‘L’ for oil type, the user can then press the second button (215b) to select a preset odor strength of the active material liquid, indicated by ‘_’ for low odor, ‘=’ for a stronger odor, and ‘≡’ for the strongest odor, as displayed on the 7-segment display (221). This setting configures the number of pulses at which the piezoelectric engine will operate using the prior ‘t’, or oil type, setting, e.g. with a setting of ‘_’, the piezoelectric engine operates for at the ‘t’ level every 20 seconds over a 60-second period, whereas a setting of ‘≡’ operates the piezoelectric engine at the ‘t’ level for every 10 seconds over a 60-second period. The user can move to the next setting, ‘d’ for duration, by pressing the first button (215a). When the 7-segment display (221) shows ‘d’ for duration, the user can then press the second button (215b) to select a preset duration for the operation of the device, indicated by ‘1’, ‘2’, ‘3’, ‘4’, and ‘8’ on the 7-segment display (221) for the number of hours the device will operate using the programmed configuration, or the figure ‘-’ shown on the 7-segment display (221) for continuous operation. Once the user has set these configuration parameters, the user can press the first button (215a) for 3 seconds, at which time the 7-segment display (221) will show ‘E’, indicating that the device has exited the configuration mode and will now run, as defined by the parameters entered by the user, when the second button (215b) is pressed. Additionally, to confirm the device is running when the second button (215b) is pressed, the 7-segment display (221) will show three ascending lines, ‘_’, then ‘=’, then ‘≡’—on the 7-segment display (221) once, and then a red dot on the 7-segment display (221) will pulse every 5 seconds until the user either interacts with the second button (215b) to change the device settings as described above, or the device is turned off using the first button (215a) as described earlier.

Again note that the above example is illustrative only, and no limitation is imposed on the customization of the device and the interface with the device in this information. In another embodiment, the user can interface with the device via the micro-USB (Micro-B) receptacle (212) accessible via a port (212a) in the middle section (20) of the device, and can instruct the device to function similar to what is described in the illustrative example using an external controller such as a computer, smartphone, and other such controlling apparatus. In another further embodiment, the microprocessor and/or the PCB are capable of wireless communication via Bluetooth, Wi-Fi, and/or other such means. As such, similar functions as described in the illustrative example can be performed on the invention wirelessly.

With reference to FIGS. 2 and 5, the invention includes an electrical power supply to enable it to function. This may be contained within the device or this may comprise an external plug that can be connected to an external electrical power source such as a household power socket via a plug extending from the device. Alternatively, the power source can be via the micro-USB (Micro-B) receptacle (212), accessible via a port (212a) in the middle section (20) of the device, which interfaces with the PCB (220). In one preferred embodiment, the source of power is internal, and comprises one or more batteries, e.g., dry cell batteries such as ‘AA’ batteries, ‘AAA’ batteries, ‘D’ batteries, watch batteries, or rechargeable batteries such as lithium ion batteries, lithium polymer batteries, and so forth. In a more preferred embodiment, the power source is a lithium polymer battery (223), which can be fitted between the PCB (220) and the outer sleeve (214) of the middle section (20) of the housing.

With reference to FIGS. 1 to 5, the invention comprises the housing (10, 20, and 23) that contains all components, including piezoelectric engine, electronics, active material reservoir container, and mounting components thereof. In various embodiments, the housing is comprised of separate component layers. In a preferred embodiment, the housing is comprised of three sections, including a top section (10) housing the piezoelectric engine and mountings, a middle section (20) housing the electronics, power source, and active material reservoir container when attached, and a bottom housing section (23), or bottom cap, enclosing the device from the bottom and supporting the active material reservoir container. In a preferred embodiment, the middle section (20) also features a sleeve (214) for the active material reservoir container, which is accommodated by and slots into a cavity (122) feature of the bottom plate (12) of top section (10). The housing and/or various housing components can be made of a thermoplastic material such as polypropylene (PP), polyethylene terephthalate (PET), polybutylene terephthalate (PBT), polycarbonate (PC), and so forth and be injection molded; or the storage material can be made of glass, e.g., silica, borosilicate, Pyrex, aluminosilicate, and so forth; or the storage material can be made of metal, e.g., stainless steel, aluminum, and so forth; or the storage material can be made of a combination of the aforementioned materials. In various embodiments, a combination of thermoplastic materials, glass materials, and metal materials can be used to construct the housings, for which the various components of the housing are connected. In other embodiments, the housings can be encased by shell materials that are designed to fit over the assembled device housings. In a preferred embodiment, the device comprises an injection-molded housing made of polybutylene terephthalate (PBT) that is homogenously mixed with 30% glass fibre.

With reference to FIGS. 1 to 5, any connecting means can be used to connect the different housing components together, including, but not limited to, screws, magnets, adhesives, rivets, clips, welding, and so forth, along with combinations thereof. In a preferred embodiment, the top section of the housing is connected to the middle section of the housing (20) using screws (113a and 113b). The screws are attached to posts (110a and 110b) that stem from the top section of the housing (10) into ports (213a and 213a) from the middle section of the housing (20). In the same embodiment, the bottom section (23) is attached to the middle section of the housing (20) via magnets (231a, 231b, 232a, and 232b), two of which (232a and 232b) are glued into posts (230a and 230b) on the bottom section (23), and two of which (231a and 231b) are glued onto the bottom of the middle section of the housing (20).

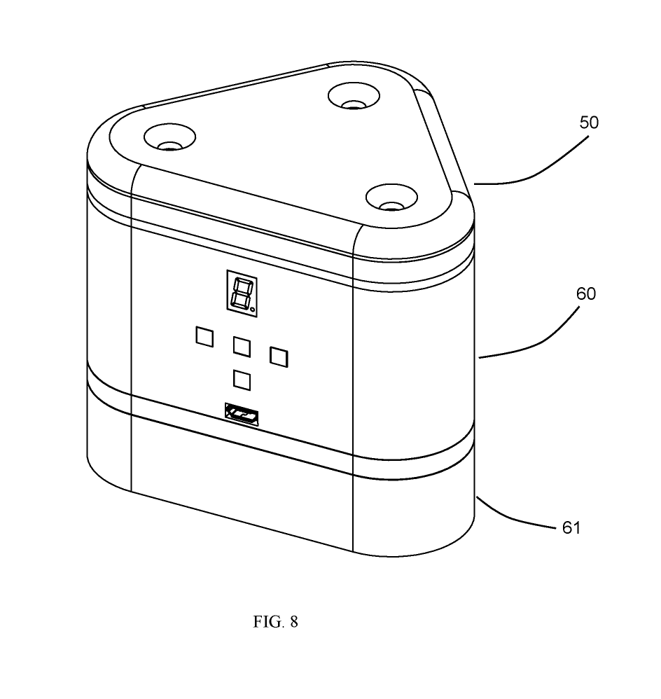

With reference to FIGS. 8 to 11, an embodiment is given comprising multiple diffusing systems, specifically three diffusing systems, in one device, hereafter referred to as the multi-system embodiment or multi-system device. There are design and systemic similarities between the single diffusing system embodiment of FIGS. 1 to 7 and the multi-system embodiment of FIGS. 8 to 11, namely that the diffusion device (FIG. 8) incorporates a top section (50) housing the diffusing engine and associated components, a middle section (60) housing the active material reservoir containers, controlling circuitry, and power source, and a bottom section (61) that supports the active material reservoir containers and encloses the device. The multi-system embodiment can use the same active material reservoir containers (40) of the single system embodiment.

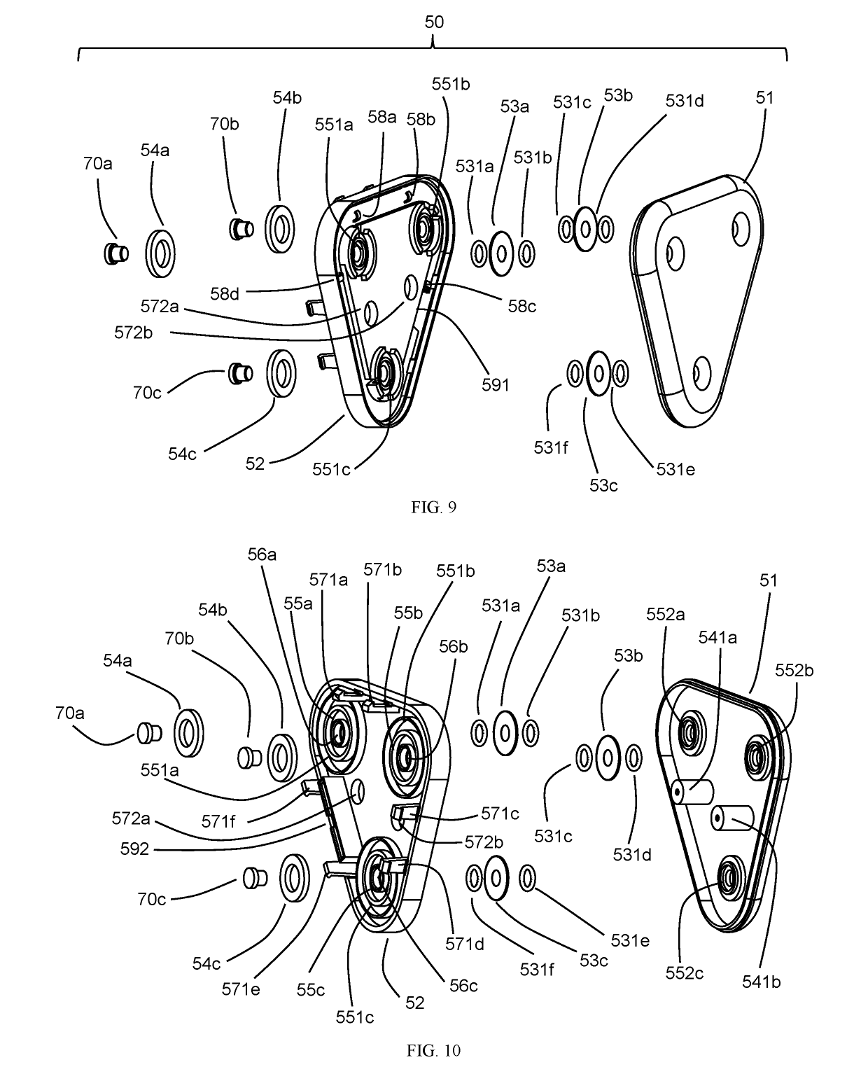

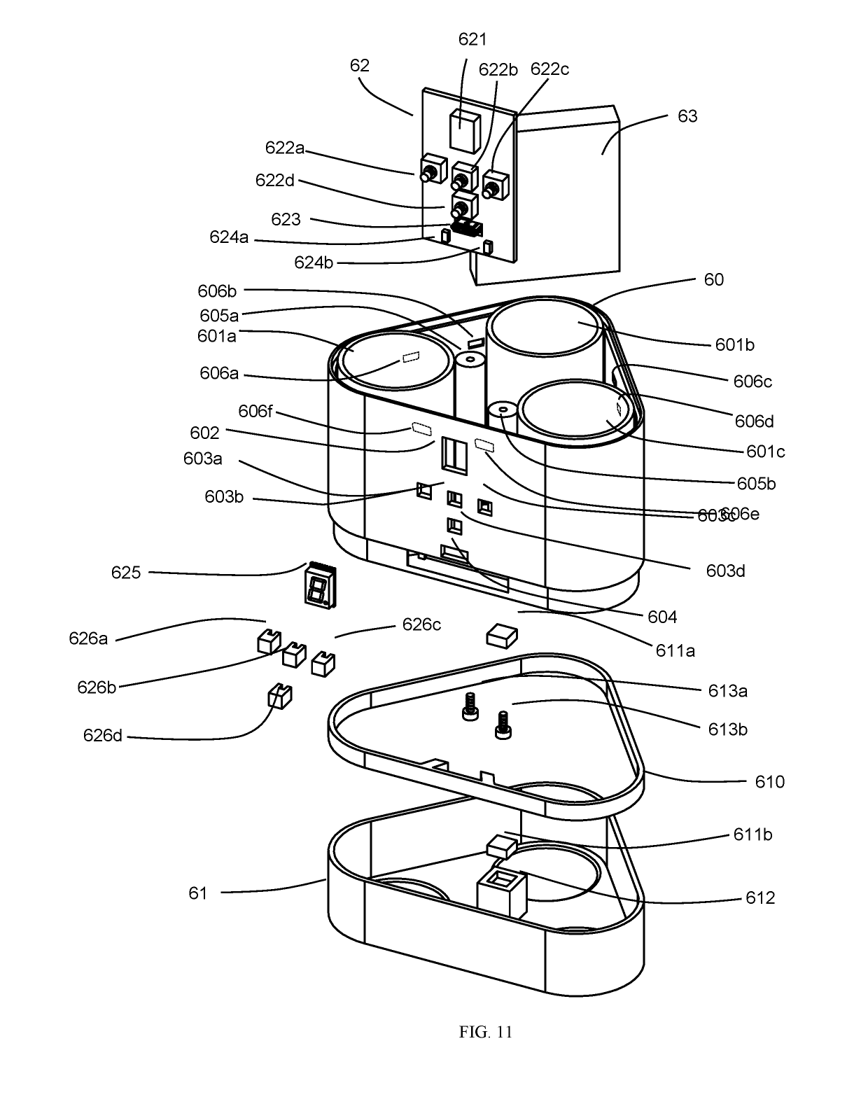

In the multi-system embodiment shown in FIGS. 8 to 11, the top section (50, and FIGS. 9 and 10) of the invention houses three piezoelectric diffusing engines (53a, 53b, and 53c), and includes the last stage of active material delivery components (70a, 70b, and 70c) along with ring-shaped magnetic docking components (54a, 54b, and 54c). The bottom section of the device (60, 61, and FIG. 11) can house up to three active material reservoir containers (FIG. 6, containers not shown in FIG. 11) in sleeved ports (601a, 601b, and 601c). The middle section of the device (60, 61, and FIG. 11) also houses components for powering the device using external power via the receptacle (623) that is accessible via a port (604) in the middle section (60) of the device, and/or using an internal battery (63), as well as components for controlling the device using a microprocessor on a PCB (62), either via a direct mechanism using buttons (626a to 626d) and 7-segment display (621, transmitted to the user via a light pipe, 625) or via an indirect mechanism such as Bluetooth, Wi-Fi, and so forth.

With reference to FIGS. 9, 10, and 11, the top section (50) of the multi-system embodiment comprises a top plate (51) and a bottom plate (52). The top plate (51) encloses three piezoelectric engines (53a, 53b, and 53c), each of which is mounted using top O-rings (531b, 531d, and 531e) placed in cavities (552a, 552b, and 552c) on the top plate, and with bottom O-rings (531a, 531c, and 531f) placed in cavities (551a, 551b, and 551c) on the bottom plate (52) of the top section (50). The O-rings can be comprised of the same materials as described for the single system embodiment shown in FIGS. 1 to 5 and described earlier. The piezoelectric engines are connected via wires (not shown) to the PCB (62), which is mounted in the middle section (60), through a rectangular gap (591) in the bottom plate (52). The wires (not shown) run along a channel of the bottom plate (52) and are secured by wire clips in the channel (58a to 58d). With reference to FIGS. 9, 10, and 11, the top and bottom plates are joined together by posts (541a and 541b) that slot into ports (572a and 572b) in the bottom plate (52), and are attached to the middle section of the housing (60) by screws (613a and 613b) that are fitted through posts (605a and 605b) in the middle section of the housing. In addition, the bottom plate (52) of the top section (50) of the multi-system embodiment also includes clips (571a to 571f) that connect the top section and the middle section of the housing more securely. These clips (571a to 571f) are attached to slots (606a to 606f; note that 606a, 606d, 606e, and 606f are represented with dashed lines as they are obscured by the oblique view of FIG. 11) in the middle section to ensure a tight fit between the top and middle sections.

With reference to FIG. 10, the last stages of the active material delivery mechanisms of the multi-system embodiment comprise three rigid, porous, hydrophilic, and oleophilic wicks (70a, 70b, and 70c) that fit into ports (56a, 56b, and 56c) on the bottom plate (52) of the top section (50). The rigid, porous oleophilic, and hydrophilic wicks are designed in the same way and are comprised of the same materials as described for the single system embodiment shown in FIGS. 1 to 5. The last stage delivery mechanisms also comprise magnetic docking mechanisms for active material reservoir containers (40) to interface with the last stage delivery components, and uses ring-shaped magnets (54a, 54b, and 54c) that slot into cavities (55a, 55b, and 55c) on the bottom plate (52) of the top section (50) of the device. This magnetic docking mechanism is similar to the docking mechanism of the single system embodiment shown in FIGS. 1 to 5.

With reference to FIGS. 8, 10 and 11, the middle section of the multi-system embodiment (60) houses the electronics, power source, and active material reservoir containers when attached, and a bottom section (61), or bottom cap, enclosing the device from the bottom and supporting the active material reservoir containers. The middle section (60) also features three sleeves (601a, 601b, and 601c) for three active material reservoir containers, which are accommodated by and slot into cavities (551a, 551b, and 551c) on the bottom plate (52) of top section (50). With reference to FIGS. 10 and 11, the PCB is mounted vertically along the back face of the middle section (60) of the device, and is supported by a placeholder (592) in the bottom plate (52) of the top section (50) of the device. The PCB (62) features a microprocessor that can be controlled via a direct mechanism using buttons (626a to 626d), fitted into ports (603a to 603d) in the middle section (60), that interface with the PCB (62) via switches (622a to 622d), along with a display interface comprised of a 7-segment display (621) and a light pipe (625), both of which fit into a slot (602) of the middle section (60). The PCB also has a receptacle (623) that is accessible via a port (604) in the middle section (60) of the device for the purpose of charging and/or communicating with the device, which can be of any appropriate charging and/or communication technology as described previously in the single system embodiment. In a preferred embodiment, the receptacle is a micro-USB (Micro-B) receptacle (623) accessible via the port (604) in the middle section (60) of the device. As with the single system embodiment, when the device is in operation, two LEDs (624a and 624b) will activate. A triangular-shaped light diffuser (610) transmits the light emitted by these LEDs (624a and 624b) as a glowing band around the perimeter of the device. As with the single system embodiment, the ring-shaped light diffuser (610) can be made of frosted glass or frosted clear plastic such as frosted polyethylene, frosted polycarbonate, and the like. With reference to FIG. 11, the multi-system embodiment includes an electrical power supply to enable it to function, which can be provided via the receptacle (623) that interfaces with the PCB (62) and is accessible via a port (604) in the middle section (60) of the device, or via a lithium polymer rechargeable battery (63). With further reference to FIG. 11, the bottom section (61), or bottom cap, is attached to the middle section (60) via magnets (611a and 611b), one of which (611b) is glued into a post (612) on the bottom section (61), and the other of which (611a) is glued onto the bottom of the middle housing (60).

Regarding the operation of the multi-system embodiment, the device can be operated in a manner similar to that described in the example given for the single system embodiment. With reference to FIGS. 9, 10, and 11, an example of the operation of the multi-system embodiment is given here; note that this example is for illustrative purposes only, and by no means limits the functionality of device in any way whatsoever. Importantly, the use of the device and the interface parameters can be customized, altered, changed, updated, and modified at any time by changing or updating the microprocessor program. In this illustrative example of a preferred embodiment, the user is able to turn the device on by holding the bottom button (626d, which activates switch 622d) firmly for 3 seconds. The device is switched on when the 7-segment display (621, transmitted to the outside of the device via a light pipe, 625, for the user to see) shows the characters ‘o’ and ‘n’ successively. When the device is on, the device can be turned off also by holding the bottom button (626a) firmly for 3 seconds, and the device is switched off after the 7-segment display (621) shows ‘o’, ‘F’, and ‘F’ successively. Once the device has been turned on, the user can set the run-time duration of each of the three piezoelectric engines and the frequency of repeat run-time operations of each engine over a preconfigured time period, as set by the microprocessor program. The configuration of each piezoelectric engine can be addressed by one of the top three buttons (626a, 626b, and 626c), with each button corresponding to an individual piezoelectric engine, i.e., button 626a corresponds to piezoelectric engine 53c, button 626b corresponds to piezoelectric engine 53a, and 626c corresponds to piezoelectric engine 53b. The user enters the configuration mode for each piezoelectric engine by holding the respective button, after which the 7-segment display (621) shows ‘C’ for the corresponding engine. The user is then able to set the operation of the selected piezoelectric engine as described in the illustrative example given for the single system embodiment, the details of which were given prior. Once the user has set the configuration parameters for the selected piezoelectric engine, the user can press the corresponding button for 3 seconds, at which time the 7-segment display (621) will show ‘E’, indicating that the device has exited the configuration mode for that piezoelectric engine, which will now run, as defined by the parameters entered by the user, when the corresponding button is pressed. Additionally, to confirm the device is running when the bottom button (626d) is pressed, the 7-segment display (621) will show three ascending lines, ‘_’, then ‘=’, then ‘≡’—on the 7-segment display (621) once, and then a red dot on the 7-segment display (621) will pulse every 5 seconds until the user either interacts with the button corresponding to the active piezoelectric engine to change the device settings as described above, or the device is turned off using the bottom button (626d) as described earlier.

As with the single system embodiment, note that the above example is illustrative only, and no limitation is imposed on the customization of the multi-system embodiment and the interface with the device in this information. In alternative embodiments, the user can interface with the device via the receptacle (623), and can instruct the device to function similar to what is described in the illustrative example using an external controller such as a computer, smartphone, and other such controlling apparatus. In another further embodiment, the microprocessor and/or the PCB are capable of wireless communication via Bluetooth, Wi-Fi, and/or other such means. As such, similar functions as described in the illustrative example can be performed on the invention wirelessly.

As with the single system embodiment of FIGS. 1 to 5, the housing and/or various housing components of the multi-system embodiment (FIG. 8) can be made of materials as described for the single system embodiment e.g., a thermoplastic material such as polypropylene, polycarbonate, polyethylene terephthalate, and so forth, or a metal such as stainless steel or aluminum or the like. As with the single system embodiment, a combination of thermoplastic materials and metal materials can be used to construct the housings, for which the various components of the housing are connected. Additionally, the housings can be encased by shell materials that are designed to fit over the assembled device housings e.g., the device can comprise an inner housing made of a thermoplastic material that is sleeved by a metal shell that encases the thermoplastic material inner housing.

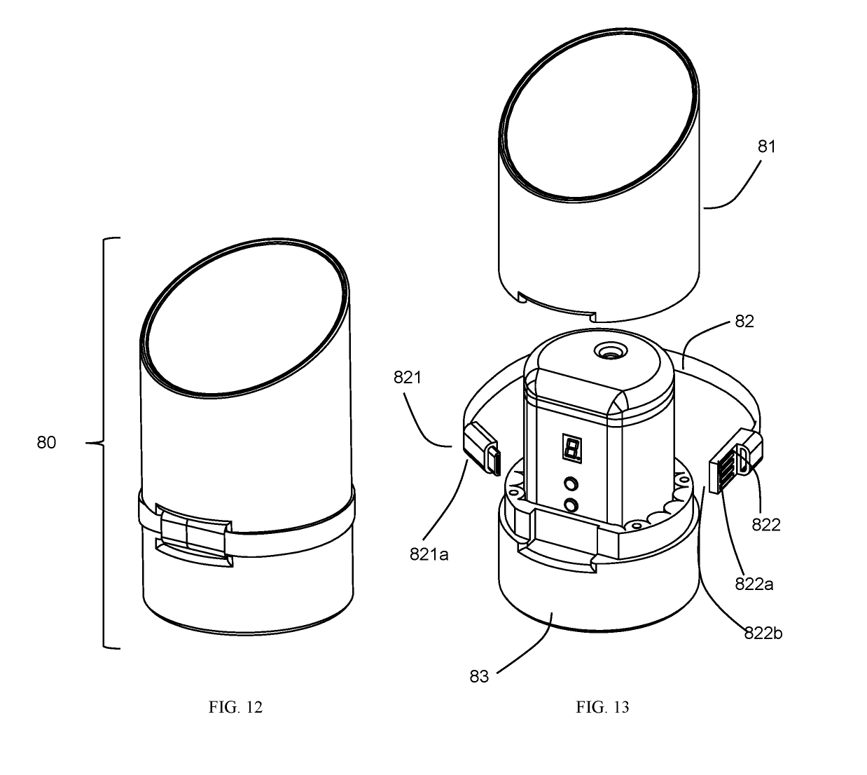

With reference to FIGS. 1, 2, 5, and 12 to 15, the invention also comprises a case (80), shown assembled and closed in FIG. 12, which can entirely house the complete single diffusing system embodiment shown in FIG. 1. The case (80) comprises three separate components that are shown in FIG. 13, with the components being a top cover (81), a charging and communication cable that wraps around the middle of the case (82), and a bottom cover (83) that supports the single diffusing system embodiment of the present invention. Regarding the charging and communication cable (82), the first end of the cable (821) features a plug (821a) that can be placed into the receptacle of the single system embodiment (212 of FIGS. 2 and 5), while the second end of the cable (822) features a plug (822b) that can fit into a compatible receptacle on an external device e.g., a computer, a charging station, a mobile phone, and so forth. In a preferred embodiment, the first end of the cable (821) features a micro-USB (Micro-B) plug (821a), while the second end of the cable (822) features a standard USB (Type-A) plug (822b). The second end of the cable (822) also features a slot (822a) that can house the plug feature (821a) of the first end of the cable (821); as such, the cable can form a band around the case (80) when the cable (822) is wrapped around the case (80) and the first (821) and second (822) ends are joined together. The case components, specifically the top (81) and bottom (83) sections, can be made of a thermoplastic material such as polypropylene, polycarbonate, polyethylene terephthalate, poly(methyl) methacrylate, and so forth, and be injection molded. Alternatively, the case components, specifically the top (81) and bottom (83) sections, may be made of metal such as stainless steel or aluminum or the like. In a preferred embodiment, the top (81) and bottom (83) sections of the case are made of clear and transparent poly(methyl) methacrylate, such that, when the single system embodiment is house within the case, it can still be viewed by the user through the case. Regarding the charging and communication cable (82), this cable can be made of a flexible plastic material (e.g., polyethylene, polypropylene, and so forth) enclosing the wiring required for communication between the first (821) and second (822) ends of the cable. The charging and communication cable can be of a length between 10 cm and 80 cm, more preferably between 20 cm and 60 cm, and be colored. In a preferred embodiment, the length of the charging and communication cable is 24.2 cm, which ensures that the cable fits securely around the middle of the case when assembled (80), and is white in color.

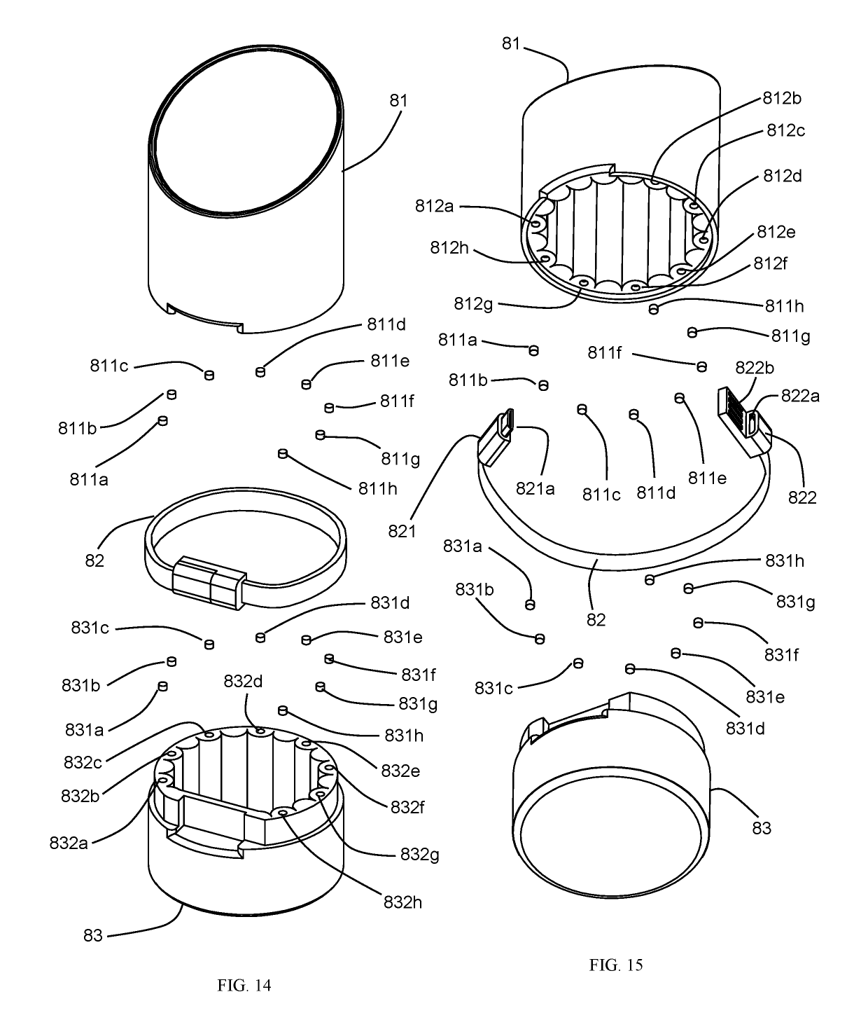

With reference to FIGS. 13, 14, and 15, the top (81) and bottom (83) sections of the case (80) can be connected together using a multiplicity of mechanisms, including, but not limited to, screws, magnets, clips, posts, bands, catches, and so forth. In a preferred embodiment shown in FIGS. 14 and 15, the method of attachment of the top (81) and bottom (83) sections of the case (80) is magnets. In this embodiment, the top section (81) of the case houses eight magnets (811a to 811h) that are fitted into ports (812a to 812h) on the bottom edge of the top section (81). In turn, the bottom section (83) of the case houses eight magnets (831a to 831h) that are fitted into ports (832a to 832h) on the top edge of the bottom section, which are oppositely aligned to the eight magnets (811a to 811h) that are housed in the bottom edge of the top section (81). As such, the top section (81) and the bottom section (83) of the case can be magnetically connected together, while the charging and/or communication cable (82) can be fitted around the outside of the case by wrapping the cable around the case and connecting the two ends (821 and 822) together.

In conclusion, the portable diffuser device of the present invention enables the diffusion and aspiration of active materials, with particular regards to liquid active materials, as aerosols using a piezoelectric engine, or, in the case of a multi-system device, multiple piezoelectric engines. The invention incorporates a method to easily add or remove active material reservoir containers, and also utilizes a novel, staged method for the delivery of the active material to the piezoelectric engine, or respective piezoelectric engines in the case of a multi-system device, for dispersion. Furthermore, the invention incorporates a direct interface mechanism and/or a wireless interface mechanism, allowing the user to control the device via a multiplicity of mechanisms, including but not limited to, a button interface, a display interface, a Bluetooth interface via an external wireless device such as a smartphone or computer, or a Wi-Fi interface via an external device such as a smartphone or computer. The invention also incorporates a case that can be used to securely house the single system embodiment of the current invention. Note that, although particular embodiments of the invention have been described in detail in this application for the purposes of providing illustrative examples, any modifications and enhancements that can or may be made to the invention are assumed to remain within the spirit and scope of the invention. Furthermore, in view of this description in its entirety, it will be apparent to those skilled in the art that numerous modifications can be made; consequently, this illustrative description is presented for the purpose of enabling those skilled in the art to construct and use the invention as required. The exclusive rights to all modifications which come within the scope of the appended claims are reserved.

1. An active material dispersion device comprising:
a) a top section, housing one active material dispersion engine and an active material delivery mechanism to the dispersion engine;
b) a middle section, featuring a sleeve for housing one active material reservoir container and driving electronics for the active material dispersion engine, as well as active material delivery stages, upon which the top portion can be placed and interface with the middle portion;
c) an active material reservoir container that contains the active material, as well as the necessary components of the active material delivery stages required for delivery of the active material to the dispersion engine;
d) a bottom section that encloses the middle section and thus the device, and supports the active material reservoir containers when they are attached to the device;
e) a case device for housing the active material dispersion device, consisting of a top and a bottom section that fully encloses the active material dispersion device.

2. The dispersion device of claim 1, whereby a dispersing engine is mounted between the two plates of the top section and atop the last stage of the active material delivery mechanism, for which the mounts are toric rings, or O-rings, comprised of fluoroelastomer copolymer materials that have a Shore A hardness of between 45 and 55.

3. The dispersion device of claim 2, where the dispersion engine is a piezoelectric engine comprised of a porous metal mesh enclosed by a piezoelectric ceramic material, whereby a voltage can be applied to the piezoelectric ceramic material and cause the ceramic to vibrate at ultrasonic frequencies, and disperse any active material that is either in contact with or near contact to the porous metal mesh.

4. The dispersion device of claim 2, where the dispersion engine is in contact with or very near to the last stage of the active material delivery mechanism, comprising a rigid, porous, oleophilic, and hydrophilic wick made of polyethylene.

5. The dispersion device of claim 2, where the bottom plate of the top section contains a ring magnet for docking with other stages of the active material delivery mechanism.

6. The dispersion device of claim 1, where the middle section of the housing contains a printed circuit board, or PCB, that features circuitry, including microprocessor that features components enabling the user to interface with the device via an indirect mechanism such as Bluetooth and Wi-Fi, a rechargeable lithium polymer battery to supply power to the circuitry and to the piezoelectric engine for the driving of the piezoelectric engine housed in the top section of the device as described in claims 2 to 4, a direct mechanism for user interface with the device, comprising a seven-segment display and two buttons, whereby the user can program the dispersion device following the settings provided by the microprocessor on the PCB, as well as a USB interface for programming of the device and charging of the rechargeable lithium polymer battery.

7. The dispersion device of claim 1, where an active material reservoir container is a container of a certain volume made of glass, plastic, or metal, and can be fit into the housing via a sleeve as described in claim 1.

8. The dispersion device of claim 7, where the active material reservoir container also features components of the active material delivery mechanism, including a soft, hydrophilic, and oleophilic wick made of polyethylene, that fits into the bottle and delivers active material to the last stage of the active material delivery mechanism described in claim 4.

9. The dispersion device of claim 8, where the active material reservoir container features a cap through which the soft, hydrophobic, and oleophilic wick can fit through and thus interface directly through contact with the last stage of the active material delivery mechanism described in claim 4.

10. The dispersion device of claim 8, where the cap component of the active material reservoir container further features a rubber- or polymer-coated washer that interfaces with and magnetically docks to the top section of the dispersion device as described in claim 5, providing good contact with the last stage of the delivery mechanism and sealing the interface between the active material reservoir container and the docking portion of the device.

11. The dispersion device of claim 1, where the bottom section of the device features two magnets mounted on posts that dock with two magnets attached to the bottom of the middle section of the device, thus supporting the active material reservoir container and fully enclosing the device.

12. An active material dispersion device comprising:
a) a top section housing multiple (more than one) active material dispersion engines, each with their own components for their respective active material delivery mechanisms;
b) a middle section featuring multiple (more than one) sleeves for housing active material reservoir containers, allowing for multiple active material reservoir containers to be placed within and be used by the device, as well as driving electronics for the active material dispersion engines and active material delivery stages, upon which the top portion can be placed and interfaced with;
c) a bottom section that encloses the middle portion and thus the device, along with any active material reservoir containers when they are attached to the device.

13. The dispersion device of claim 12, where the top section is comprised of two plates that are connected by posts fitted through ports in the bottom plate, as well as clips that extend from the side of the top plate, and attached to the middle section via screws that are fitted through posts in the middle section, and slots for the clips.

14. The dispersion device of claim 13, where multiple (more than one) dispersing engines are mounted between the two plates of the top section and atop the last stage of the active material delivery mechanism, whereby the mounts for each of the dispersing engines are toric rings, or O-rings, comprised of fluoroelastomer copolymer materials that have a Shore A hardness of between 45 and 55.

15. The dispersion device of claim 13, where the dispersion engines are identical to the dispersion engine described in claim 3.

16. The dispersion device of claim 13, where the dispersion engines are in contact with or very near to the last stage of the active material delivery mechanism for each dispersion engine, comprising rigid, porous, oleophilic, and hydrophilic wicks made of polyethylene fiber.

17. The dispersion device of claim 13, where the bottom plate of the top section contains a ring magnet at each of the last stage delivery mechanisms, for docking with other stages of the active material delivery mechanisms.

18. The dispersion device of claim 12, where the middle section of the housing contains a printed circuit board, or PCB, that features circuitry, including microprocessor that features components enabling the user to interface with the device via an indirect mechanism such as Bluetooth and Wi-Fi, a rechargeable lithium polymer battery to supply power to the circuitry and to the piezoelectric engine for the driving of the piezoelectric engines housed in the top section of the device as described in claims 14 to 16, a direct mechanism for user interface with the device, comprising a seven-segment display and two buttons, whereby the user can program the dispersion device following the settings provided by the microprocessor on the PCB, as well as a USB interface for programming of the device and charging of the rechargeable lithium polymer battery.

19. The dispersion device of claim 12, where the bottom section of the device features one magnet mounted on a post that docks with a magnet attached to the bottom of the middle section of the device, thus supporting the active material reservoir containers placed within the device and fully enclosing the device.

20. The case device of claim 1(e), where the top and bottom sections of the case can fully enclose the active dispersion device of claim 1 and can be connected using magnets, and includes a USB cable that wraps around the center of the case device thus fully enclosing the case device and the active dispersion device of claim 1.
